(19) United States
(12) Patent Application Publication
(10) Pub. No.: US 2006/0063185 A1
(43) Pub. Date: Mar. 23, 2006

(54) Methods for detection of pathogens in red blood cells

(76) Inventor: Edouard Vannier, Boston, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/223,599

(22) Filed: Sep. 9, 2005

Related U.S. Application Data
(60) Provisional application No. 60/608,825, filed on Sep. 9, 2004.

(51) Int. Cl. C12Q 1/68; G01N 33/569; G01N 33/53

(52) U.S. Cl. 435/6; 435/7.22

(57) ABSTRACT

The present invention relates to methods for diagnosing a parasitic microorganism that resides in red blood cells, such as Babesia microti.

### RELATED APPLICATION

This application claims priority to US provisional patent application 60/608,825, filed Sep. 9, 2004, which is incorporated herein by reference in its entirety.

### FIELD OF THE INVENTION

Disclosed are methods for the diagnosis of infections caused by pathogenic agents that reside in red blood cells, such as Babesia microti.

### BACKGROUND OF THE INVENTION

Babesiosis is a tick-borne zoonosis caused by protozoa of the genus Babesia. In the United States, human babesiosis due to Babesia microti is an emerging infectious disease transmitted by the hard-bodied tick Ixodes scapularis (also known as L dammini). While infections are often subclinical, severe disease is seen in immunosuppressed individuals. In Europe, human babesiosis is rare, but often severe. On the basis of morphology and antigen reactivity, most cases have been attributed to the cattle pathogen B. divergens. In fact, some of these cases may have been due to Babesia EU1, a pathogen closely related to B. divergens. Only recently has B. microti been identified as the etiologic agent of human illness in Switzerland. B. microti infection may be underdiagnosed in central Europe since antibodies against B. microti have been detected in serum from residents of western Germany and eastern Switzerland. Ixodes ricinus, the tick that transmits B. divergens to cattle and people, is a competent vector of B. microti transmission. B. microti has been detected in I. ricinus collected in regions of Switzerland, Slovenia, Hungary and Poland.

### SUMMARY OF THE INVENTION

The invention features methods and compositions for detecting an infection of a red blood cell, e.g., a mature erythrocyte or a reticulocyte by a microorganism. A method for diagnosing the presence of a parasitic microorganism is carried out by determining a fraction of DNA-containing, transferrin receptor-negative cells in a blood sample, e.g., mammalian red blood cells, in fluid phase. An increase in the fraction (or per cent) of DNA-containing, transferrin receptor-negative cells compared to a normal fraction indicates that the mammal from which the blood sample was obtained is infected with the parasitic microorganism. The microorganism is transmitted by a vector such as a tick or a mosquito. Tick-borne pathogens include Babesia sp., e.g., Babesia microti, or Bartonella sp., and mosquito-borne pathogens include Plasmodium sp. Preferably, the detecting step is carried out by flow cytometry.

The tissue sample is a volume of peripheral blood from a mammal, e.g., a human subject, dog, wolf, coyote, cat, cow, sheep, goat, horse, deer or other animal such as a member of rodent species. The tissue sample contains erythrocytes. The sample also contains a population of reticulocytes, i.e., immature red blood cells, as a fraction of total red blood cells that changes during the course of infection. The method includes a step of identifying a population of immature red blood cells by detecting the transferrin receptor. The method for testing reticulocytes also includes a step of removal or enzymatic digestion of RNA, e.g., by contacting the cells with an enzyme such as RNase. Preferably, the RNase is purified. For example, the composition is 85%, 90%, 95%, 99% or greater than 99% RNase by weight. For example, the RNase is chromatography-grade RNase. The method includes a step of detecting expression of a CD71 antigen on the surface of red blood cells, e.g. by contacting the sample with a CD71-specific antibody. Antibodies that bind to a transferrin receptor (CD71) of numerous species are known in the art and widely available. Exemplary antibodies include a rat anti-mouse CD71 monoclonal antibody (Pharmingen, Cat#553264) followed by a secondary antibody, namely Alexa647-conjugated goat anti-rat antibody (Molecular Probes, Cat#A21247). Alternatively, any CD71 specific monoclonal antibody directly conjugated to a fluorochrome (e.g., Alexa647), or any unconjugated CD71 specific monoclonal antibody recognized by a secondary antibody conjugated to a fluorochrome is useful. Useful antibodies directed to human CD71 include RDI-CBL137 (Research Diagnostics, Inc. Flanders, N.J.) and Ab10247 and 10259 (Abcam, Cambridge, Mass.).

RNA content differs between reticulocytes and mature red blood cells. Samples are treated to degrade or destroy RNA from parasite and reduce the RNA content from reticulocytes. The method also includes a step of fixing the sample of mammalian red blood cells, e.g., by contacting the sample with glutaraldehyde. The method further comprises permeabilizing the sample of mammalian red blood cells, e.g., by contacting the sample with a detergent.

To label red blood cells that contain pathogen DNA, the method includes a step of determining the fraction of DNA-containing mammalian red blood cells by contacting the sample of red blood cells with a fluorescent nucleic acid stain, such as a dimeric cyanine nucleic acid stain. Exemplary stains include commercially-available reagents such as YOYO-1, propidium iodide, thiazole orange, SYBR Green I, SYTOX Green, Pico Green, POPO-1, BOBO-1, YOYO-1, POPO-3, LOLO-1, BOBO-3, YOYO-3, TOPRO-3 or TOTO-3. Any nucleic acid binding reagent that is fluorescent upon activation by a laser is applicable to detect DNA-containing red blood cells. Optionally, the reagent preferentially stains DNA compared to RNA.

Also within the invention is a kit for diagnosis of a parasitic microorganism in a mammalian red blood cell. The kit contains a ligand that binds to an epitope of the transferrin receptor (e.g., CD71) or other cell surface marker and a composition that binds to nucleic acids such as those found in a DNA molecule. The composition preferentially binds to a DNA molecule compared to an RNA molecule. Preferably, the ligand is an antibody, e.g., a monoclonal antibody that binds to CD71. The ligand and composition have different fluorescent probes, so as to differentially detect DNA content and transferrin receptor expression. In preferred embodiments, the DNA-detection composition is YOYO-1.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE FIGURES

FIG. 1 is a series of photographs demonstrating that Babesia antigens and DNA co-localize to mature erythrocytes, but not to reticulocytes. Blood was obtained from an infected C.B-17.scid mouse. Cells were fixed in glutaraldehyde, permeabilized in Triton X-100 and treated with 100 μg/ml DNAse-free RNAse A. Cells were stained for the transferrin receptor CD71 (FIG. 1C, green), DNA (FIG. 1B, blue) with DAPI, and Babesia antigens (FIG. 1A, red) with a polyclonal antibody obtained from a DBA/2 mouse three months after it was infected with B. microti. FIG. 1D is an overlay of FIGS. 1A-C. CD71 is present on most reticulocytes but absent from terminally differentiated erythrocytes.

FIG. 2 is a series of graphs demonstrating that nucleic acid staining is sensitive to RNAse in reticulocytes, but not in Babesia microti infected erythrocytes. Blood was obtained from an infected C.B.-17.scid mouse. Upon fixation and permeabilization, whole blood cells were treated with increasing concentrations (from 1 to 300 μg/ml) of DNAse-free RNAse A. Cells were stained for nucleic acids with YOYO-1 and for the transferrin receptor CD71 (FIG. 2A). For each RNAse A concentration, control cells were exposed to an irrelevant mAb directed against KLH (FIG. 2B). Data are representative of three separate experiments. Ovals indicate the intensity of YOYO-1 staining when CD71 positive cells were not treated with RNAse A. Note that the intensity of YOYO-1 staining in CD71 negative cells (lower right quadrants of each graph) was not affected by RNAse A treatment.

FIG. 3 is a dot graph and a series of bar graphs demonstrating that Babesia microti primarily reside in mature erythrocytes. Blood was obtained from C.B-17.scid mice three months after infection with 10⁵ pRBCs. Whole blood cells were fixed, permeabilized and treated with 100 μg/ml DNAse-free RNAse A. Cells were stained for nucleic acids with YOYO-1 and for CD71. YOYO-1+ cells were fractionated by fluorescence activated cell sorting (FIG. 3A). CD71− cells were sorted into fractions (marked by vertical rectangles) according to their content in nucleic acids. CD71+ cells were sorted as a single fraction, represented by the large horizontal rectangle. Fractionated cells were stained for the pan-erythroid surface marker TER119. Each fraction was examined under fluorescent microscopy, and the number of nuclei per cell (ranging from 0 to 8) was counted in 100 cells (boxes contained within FIG. 3A). The median number of nuclei per cell in CD71− cells ranged from one in the fraction with the lowest YOYO-1 staining (FIG. 3B, F1) to two and three in the fractions with higher intensity, namely F2 and F3, respectively (FIGS. 3C and 3D). CD71+ cells were rarely positive for nuclei (FIG. 3E, F4). Data are mean±SEM for cells from three mice.

FIG. 4 is a dot graph and a series of photographs showing budding and multiple infections in Babesia microti-infected erythrocytes. Blood cells from Babesia-infected C.B-17.scid mice were fractionated on the basis of CD71 surface expression and nucleic acid content (FIG. 4G). Nucleic acids were stained in green (YOYO-1) whereas the surface marker TER119 was red (Alexa 594). In CD71+ cells (FIG. 4A), the uniform distribution of numerous tiny dim green dots reflected the residual RNA content, despite RNAse treatment. These tiny dots were not seen in CD71− cells (FIGS. 4B-F). In these cells, parasite nuclei appeared as bright large green dots. As YOYO-1 staining became brighter, the number of nuclei per cell increased (FIGS. 4B-F). In CD71− cells with intense YOYO staining, nuclei varied in number (FIGS. 4C-F) and size (FIG. 4E). Some nuclei were in close proximity, indicating binary fission (FIG. 4B). Other nuclei were distant from each other, indicating multiple infections per cell (FIGS. 4C-E). The majority of cells in the brightest YOYO-1 staining (far right fraction) contained four or more nuclei (data not shown), thereby increasing the chances of visualizing four daughter cells arranged in a tetrad or Maltese cross (FIG. 4F).

FIG. 5 is a series of line graphs demonstrating that reticulocytes remain refractory to Babesia microti despite severe host susceptibility. Mice from the DBA/2 (n=4) and C.B-17.scid (n=3) strains were infected by intraperitoneal injection of 10⁵ pRBCs (day 0). One additional mouse of each strain served as an uninfected control. Blood samples were obtained at two-to-four day intervals from day 10 to day 31. (FIGS. 5A and 5C) On each of these days, a drop of blood was placed on a glass slide, a thin blood smear obtained and nuclear material revealed by Giemsa stain. A second drop of blood was placed in heparinized PBS. Blood cells were stained for Babesia antigens (Bab pAb), nucleic acids (YOYO-1), and CD71. (FIGS. 5B and 5D) CD71+ cells were analyzed for nucleic acid content and Babesia antigen expression throughout the course of infection. For each day, staining for the uninfected mouse was subtracted from the staining of each infected mouse. Data are mean±SEM of stained cells as percent of total counted cells. Note that nearly all CD71+ reticulocytes in DBA/2 and C.B-17.scid mice failed to express Babesia antigens. Despite RNase A treatment, YOYO-1 stained residual RNA in reticulocytes, as illustrated in FIG. 3, panel A.

FIG. 6 is a set of line graphs demonstrating that frequency of YOYO+CD71− cells is an accurate measure of parasitemia in Babesia microti infected mice. DBA/2 (FIG. 6A) and C.B-17.scid (FIG. 6B) mice were infected with B. microti. Infection was monitored from day 10 to day 31. Parasitemia defined as the frequency of infected red blood cells assessed by microscopic analysis of Giemsa stained blood smears was tested for an association with the frequency of YOYO+CD71− cells (open squares) or Babesia antigen positive cells (closed triangles) determined by flow cytometry. Coefficients of correlation are reported as r². Slope and origin are reported for each linear regression.

FIG. 7 is a series of line graphs showing early reticulocytosis in resistant mice, but delayed and sustained reticulocytosis in the absence of adaptive immunity. C.B-17.scid (FIG. 7A; n=4), C.B-17 (FIG. 7B; n=4), BALB/cBy (FIG. 7C; n=4) and B10.D2 (FIG. 7D; n=7) mice were infected by intraperitoneal injection of 10⁵ pRBCs (day 0). One additional mouse of each strain served as an uninfected control. A drop of blood was collected in heparinized PBS at two-to-four day intervals from day 7 until day 91 (FIGS. 7A-C) or day 79 (FIG. 7D). Blood cells were stained for nucleic acids (YOYO-1), and for CD71. For each day, staining for the uninfected mouse was subtracted from the staining of each infected mouse. Data are mean±SEM of stained cells as percent of total counted cells.

FIG. 8 is a diagrammatic representation of methods for assessing parasitemia.

FIG. 9 is a line graph showing the predicted US population over the age of 65 from 1960 until 2050. Age is the most-heavily weighted variable determining clinical outcome of infection. Currently, infection causes 40% of all mortality in individuals over the age of 65 years.

FIG. 10 is a diagram illustrating the life cycle of Babesia microti.

FIG. 11 is a diagram illustrating the transmission of Babesia microti.

FIG. 12 is a diagrammatic representation of a Babesia microti infection protocol.

FIGS. 13A-C are line graphs demonstrating that BALB/c mice are highly resistant to Babesia microti infection.

FIGS. 14A-C are line graphs demonstrating that C57BL/6 mice are highly resistant to Babesia microti infection.

FIGS. 15A-C are line graphs demonstrating the increased susceptibility of aged DBA/2 mice to Babesia microti infection.

FIG. 16 is a line graph demonstrating the measurement of parasite burden.

FIGS. 17A and 17B are a set of line graphs demonstrating the age-dependent susceptibility to Babesia microti infection in DBA/2 mice.

### DETAILED DESCRIPTION OF THE INVENTION

Babesia species are obligate parasites of red blood cells. Following invasion, Babesia sporozoites and merozoites evolve into trophozoites that move freely in the host cell cytoplasm. Asynchronous, asexual budding of a trophozoite generates two to four daughter cells, or merozoites. Because egress of merozoites is accompanied by lysis of the host cell, anemia and reticulocytosis are two of the clinical features of severe babesiosis. Babesia species differ in their tropism for red blood cells. For instance, the murine B. hylomysci has a tropism for mature erythrocytes, whereas the canine B. gibsoni preferentially multiplies in reticulocytes. B. microti have been visualized in mouse reticulocytes.

In patients, Babesia microti is routinely detected by microscopic analysis of Giemsa-stained thin blood smears. The extent of infection is typically determined by analysis of 100 to 500 red blood cells located in few microscopic fields selected at the “feather” of the smear. Flow cytometric assays assess the viability and growth of B. bovis in red blood cells in vitro, and to quantify the percentage of red blood cells infected with B. canis or B. gibsoni in naturally or experimentally infected dogs. A new mouse model of infection with B. microti has recently emerged. Using a clinical isolate maintained in ticks, it was observed that DBA/2 mice develop an intense but transient parasitemia, whereas C57BL/6 and BALB/c mice present a marginal parasitemia. Using scid mice which lack T and B lymphocytes, it was confirmed that adaptive immunity is required for a sustained resistance to babesiosis in BALB/c mice.

The present invention uses art-recognized models of B. microti infection to ascertain the contribution of reticulocytes and erythrocytes to the parasite burden. A flow cytometric assay is disclosed that uses a sensitive nucleic acid dye such as YOYO-1 and the detection of the transferrin receptor, a surface antigen expressed by reticulocytes, but not by terminally differentiated erythrocytes. Prior to the invention, detection of Babesia microti in red blood cells was routinely carried out by microscopic analysis of Giemsa-stained thin blood smears. This technique is labor intensive and subjective. Typically, a laboratory technician counts the number of infected red blood cells among 100 to 500 cells located in a few microscopic fields selected at the “feather” of the smear. Each evaluation may take up to 1 minute. Other techniques have been developed, including ELISA and PCR. ELISA measures levels of antibodies directed against Babesia antigens. Because antibodies are detected in the circulation even after resolution of infection, ELISA does not discriminate between on-going and resolved infections. Moreover, in the case of fulminant babesiosis where infection develops before circulating antibody titers rise, ELISA can not reveal early infection. On the other hand, PCR detects the overall presence of Babesia DNA in a blood sample, but provides information neither on the number of infected red blood cells nor on the number of parasites per cell.

The methods disclosed herein utilize a fluid phase evaluation technique, e.g., flow cytometry, to detect simultaneously DNA content and CD71, the transferrin receptor. DNA content in red blood cells is measured upon excitation of a fluorescent nucleic acid dye such as YOYO-1. Staining by YOYO-1 requires prior permeabilization of the red blood membrane and of parasite membranes by exposure to a detergent such as Triton X-100. When using only DNA staining, the method yielded results similar to those of Giemsa-stained blood smears in the early period of infection, when reticulocyte counts are low. At later stages of infection, reticulocytosis develops as a consequence of red blood cell lysis. Data indicated that reticulocytes do not express babesial antigens, because they are rarely infected with Babesia microti. Immature reticulocytes, unlike mature red blood cells, contain high levels of RNA. Despite treatment with DNase-free RNase, reticulocytes stain with YOYO-1. Reticulocytes were identified as a source of false positive staining. Thus, the staining attributed to reticulocytes was excluded on the basis of expression of CD71, a surface marker that is absent from mature red blood cells. Parasitemia is expressed as the percentage of YOYO+, CD71-cells. This data strongly correlates with parasitemia defined as the percentage of infected red blood cells visualized on Giemsa-stained blood smears. More importantly, the absolute number of infected red blood cells generated by those techniques are equivalent.

The invention provides a high throughput method to quantify parasitemia in the blood of Babesia microti-infected laboratory mice. This method uses the dual detection of babesia DNA and host CD71, and may be of use for any infection with Babesia species.

Babesia sp. Infection

Age is by far the most heavily-weighted variable determining clinical outcome of infection. Infection causes 40% of all mortality in individuals >65 years of age

Babesia microti has been detected in Nantucket, Martha's Vineyard, Cape Cod (Mass.), Block Island (R.I.), eastern Long Island, Shelter Island and Fire Island (N.Y.), coastal areas of northeastern US states (Connecticut, New Jersey) as well as Georgia, Virginia, Maryland and even Taiwan. Serological evidence of B. microti infection has been identified in Germany and Switzerland. Babesia divergens is responsible for most of the cases in Europe. Rare cases with B. divergens-related organisms have been detected in the US: Missouri (MOI), Kentucky.

Infected young healthy subjects exhibit subclinical or mild infection (“summer flu”). Young immunocompromised patients exhibit severe disease in HIV, transplant and asplenic patients, whereas otherwise healthy individuals aged 50 and above often exhibit severe disease. Age-related susceptibility is also seen in other species, e.g. cattle, dogs. There is a model of age-acquired susceptibility to B. bovis in cattle, but B. bovis is not pathogenic to humans. Prior to the invention, there was no model of age-acquired susceptibility to B. microti in inbred, genetically-defined mice.

Susceptibility of Inbred Strains

Control of parasitemia in DBA/2 mice is poor. Control of parasitemia in C57BL/6 (and BALB/c) mice is excellent. Genetic variations affect resistance to B. microti. Late parasitemia in DBA/2 mice dramatically increases with age. Late parasitemia in C57BL/6 (and BALB/c) mice does not increase with age. Differences in susceptibility to B. microti between strains and age groups suggest a polygenic regulation

Infection of Red Blood Cells with Bartonella ssp.

Certain species of ticks play a role in the transmission of the bacteria Bartonella spp. to humans. Reservoirs include domesticated cats (Felis domesticus, Felis catus). Vectors include Cat Flea (Ctenocephalides felis), Body Louse (Pediculus humanus corporis), and Tick species (Ixodes spp. & Dermacentor spp.). The causative agent includes Bartonella bacilliformis, Bartonella Quintana, and Bartonella henselae (cat scratch disease). Infection is reliably detected using the methods described herein.

Identification of Loci for Resistance to Babesia

Babesiosis is an emerging infectious disease in New England, mainly seen in immunocompromised patients and in healthy individuals over the age of 50. To determine the genetic basis of susceptibility, a mouse model of infection was developed with a human isolate of B. microti. Mice were infected i.p. with 10⁵ parasitized red blood cells. Parasitemia and reticulocytosis were monitored by flow cytometry analysis of fixed and permeabilized whole blood cells. Reticulocytes were identified as CD71+ cells. The B. microti isolate infects only mature red blood cells. Parasitemia was defined as the percentage of CD71− cells that stained with the nucleic acid dye YOYO-1. DBA/2 mice developed intense parasitemia and reticulocytosis. Neither parasitemia nor reticulocytosis was detected in B10.D2 mice. As B10.D2 and DBA/2 mice share the major histocompatibility (MHC) haplotype H2D, MHC alleles are not the basis for the difference in resistance. Male mice from reciprocal (DBA/2×B10.D2) F1 mice developed neither parasitemia nor reticulocytosis, indicating that resistance is a dominant trait conferred by autosomal genes. Segregation analyses of 141 informative male F2 mice mapped a major locus of resistance to parasitemia (Babesiosis resistance locus-1, Brl-1; LOD 13.9) and reticulocytosis (LOD 16.2) on the proximal region of chromosome 9 that accounted for 38% and 41% ofthe respective phenotypic variance. A weaker linkage to parasitemia was detected on distal chromosome 4 (Brl-2; LOD 4.5). Another locus on distal chromosome 7 (LOD 3.5) affected reticulocytosis. F2 mice homozygous for B10 alleles at Brl-1 and Brl-2 were more susceptible to parasitemia than B10.D2 parents whereas F2 mice homozygous for D2 alleles at these loci were more resistant than DBA/2 parents, suggesting that additional loci contribute to resistance to parasitemia. These studies establish that B10.D2 alleles at Brl-1 and Brl-2 are determinants of resistance to B. microti.

### EXAMPLE 1

### Demonstration of Preferential Invasion of Murine Erythrocytes by Babesia microli

The following materials and methods were used to generate the data described in Example 1.

Mice. DBA/2 and B10.D2 mice were purchased from Jackson Laboratories (Bar Harbor, Me.). B10.D2 mice are C57BL/10 mice that are congenic for the MHC locus (haplotype H^{2d}) obtained from the DBA/2 strain. BALB/cBy mice were purchased from the National Institute on Aging whereas C.B-17 and C.B-17.scid mice were purchased from Taconic, Inc. (Germantown, N.Y.). C.B-17 mice are BALB/c mice congenic for the immunoglobulin heavy chain Igh^{b} allele obtained from the C57BL/Ka strain. In addition to the Igh^{b} allele, C.B-17.scid mice carry the spontaneous mutation scid, which prevents differentiation of T and B cells. All mice were maintained under specific-pathogen free conditions in clean well-tendered quarters. Mice were provided with water and chow ad libitum.

Infection of mice with Babesia microti. C.B-17.scid mice were exposed to 5-10 Ixodes scapularis nymphs infected with RMJNS, an isolate obtained in 1997 from a Nantucket Island resident diagnosed with babesiosis. Parasitemia was monitored by analysis of Giemsa-stained blood smears starting fifteen days after ticks had detached from their hosts. When 1 to 35% of RBCs were infected (sigmoid growth), blood was collected into Alsever's solution and diluted in PBS. Mice were injected with 10⁵ pRBCs delivered in 0.2 ml PBS by intraperitoneal injection.

Polyclonal Antibody to B. microh. A polyclonal antibody directed against B. microti antigens was obtained by terminal bleeding of a DBA/2 mouse that had been infected with B. microti for three months. Whole blood was collected on EDTA, and platelet-poor plasma separated by centrifugation at 4° C. A non-immune plasma was obtained from an uninfected DBA/2 mouse.

Flow Cytometry. At two-to-four day intervals, a drop of blood was obtained by tail snipping and was collected in 250 μl PBS containing 16 IU/ml heparin. Cells were fixed in glutaraldehyde (0.00625%) for 30 min at room temperature, permeabilized in Triton X-100 (0.25%) for 5 min at room temperature, and treated with 100 μg/ml heat-inactivated pancreatic RNAse A (Roche Diagnostics, Indianapolis, Ind., or chromatography grade RNase, available from Sigma) for 15 min at 37° C. Following centrifugation, cells were resuspended in staining buffer, i.e., PBS containing 1% normal rabbit serum and 0.1% sodium azide. For each sample, cells were split into two reaction tubes. In the first series of tubes, cells were stained for 30 min at room temperature with 0.5 μg/ml of rat IgG1 monoclonal antibody directed against mouse CD71, the transferrin receptor (BD Biosciences, San Jose, Calif.). In the second series of tubes, cells were incubated with a rat IgG1 monoclonal antibody directed against keyhole limpet hemocyanin, an irrelevant antigen (BD Biosciences). Upon completion of primary staining, cells were washed and resuspended in staining buffer. In all reaction tubes, cells were stained in 50 μl for 20 min at room temperature with 1.25 μg/ml of Alexa 647-labeled goat anti-rat IgG whole antibodies (BD Biosciences). The reaction volume was brought to 500 μl and the nucleic acid dye YOYO-1 iodide (1 μl in DMSO; final concentration 20 nM; Molecular Probes, Eugene, Oreg.) was added to the first series of reaction tubes. The second series of tubes was left alone as 0.2% DMSO does not affect the fluorescence of cells stained for the transferrin receptor or keyhole limpet hemocyanin. All tubes were incubated for at least 60 min at room temperature while protected from light under aluminum foil. Fluorescence was detected with a FACSCalibur (Becton Dickinson, San Jose, Calif.) using CellQuest. Upon excitation by the Argon-Ion laser at 488 nm, the nucleic acid dye YOYO-1 emits at 509 nm. Upon excitation by the He—Ne laser at 633 nm, the fluorochrome Alexa 647 emits at 669 nm. The distance between the two lasers was calibrated at each use of the FACSCalibur. Fluorescence emitted in FL1 and FL4 was analyzed using the WinMDI software.

In some experiments, as indicated, cells were stained for nucleic acids, CD71, and Babesia antigens. In the first staining step, cells were exposed to the rat anti-mouse CD71 mAb (or its isotype control) and to immune plasma containing B. microti specific antibodies (or to non-immune plasma as control). In the second staining step, cells were incubated with Alexa 647-conjugated goat anti-rat IgG and with a biotin-conjugated goat anti-mouse IgG adsorbed with rat IgG (Southern Biotechnology Associates Inc., Birmingham, Ala.). In the third staining step, cells were exposed to PerCP-streptavidin (0.125 μg/ml; BD Biosciences). For single stain controls, whole blood cells were obtained from a C.B-17.scid mouse infected with B. microti for more than four weeks, i.e., presenting high and sustained parasitemia levels. Upon proper compensation using the single stains, fluorescence emitted in FL1 upon excitation of YOYO-1 reflected nucleic acid content, whereas fluorescence emitted in FL3 (PercP) and FL4 (Alexa 647) reflected the expression of Babesia antigens and CD71, respectively. Compensation was set prior to acquisition by CellQuest, and during analysis with the Winlist software.

Assessment of Parasitemia by Microscopy. A drop of blood collected at the tip of the tail was placed on a precleaned microscope glass slide (Fisher Scientific, Pittsburgh, Pa.). A thin blood smear was obtained, quickly air-dried, and fixed in anhydrous methanol for 1 min. Smears were exposed for 60 min at room temperature to Giemsa stain diluted in PBS. Stained smears were rinsed thoroughly in water, air-dried and read under oil immersion at a magnification of 1000×. Counting was performed by an individual blinded to the source of each sample. Parasitemia was expressed as the number of erythrocytes containing at least one ring form (trophozoite or merozoite) per 100 erythrocytes analyzed. When parasitemia was below 1%, a second set of 100 erythrocytes was analyzed.

Immunofluorescence of Babesia microti infected red blood cells. The first step for staining of CD71 and Babesia antigens is described above under “Flow Cytometry”. In the second step, cells were incubated with Alexa 488-conjugated goat anti-rat IgG (Molecular Probes) and with the biotin-conjugated goat anti-mouse IgG. In the third step, cells were incubated for 10 min with Alexa 594-streptavidin (0.125 μg/ml; Molecular Probes). Cells were washed and resuspended in staining buffer containing DAPI (6.7 μM; Molecular Probes). Cells were incubated in the dark for 10 min, spun and resuspended in staining buffer. Cells were placed on a precleaned microscope slide, covered with a glass coverslip and analyzed on a Nikon Eclipse E400 fluorescence microscope under immersion oil at 1000× or 2000×. Images were captured using the Spot Advanced software. CD71 was visualized in green, Babesia antigens in red, and DNA in blue.

Fractionation of Babesia microti infected red blood cells. Whole blood cells were stained for detection of CD71 and nucleic acids (see “Flow Cytometry”). Cells were sorted at room temperature on a MoFlo (DakoCytomation, Fort Collins, Colo.). YOYO-1 positive, CD71 negative cells were sorted into several fractions. An additional fraction consisted of YOYO-1 positive, CD71 positive cells. Sorted cells were spun, and exposed for 30 min at room temperature to 0.5 μg/ml of a biotin-conjugated rat IgG_{2b} directed against the mouse pan erythroid surface marker TER-119 (BD Biosciences). Cells were washed and incubated for 10 min with Alexa 594-streptavidin (0.125 μg/ml). Cells were incubated in the dark for 10 min, spun and resuspended in staining buffer. Cells were analyzed on a Nikon Eclipse E400 fluorescence microscope.

Statistical Analysis. Data are reported as mean±SEM for the indicated number of replicates. Linear regression analysis was used to assess the relationship between frequencies of stained cells. Pearson's linear correlation coefficients were used to express the univariate relationships. Differences were considered significant if the two-tailed P values were less than, or equal to 0.05. The statistical package used for analyses was Systat 12.0 for Windows (SPSS, Inc., Evanston, Ill.).

Defining the host cells in babesiosis is important to the understanding of resistance and/or susceptibility. B. microti invasion of reticulocytes was determined by infecting the susceptible inbred mouse strains C.B-17.scid and DBA/2 with a clinical isolate of B. microti. To summarize the data detailed below, staining of fixed permeabilized red blood cells with DAPI or YOYO-1, a sensitive nucleic acid stain, revealed parasite nuclei as large bright dots. Flow cytometric analysis indicated that parasite DNA is primarily found in mature erythrocytes that expressed Babesia antigens but not the transferrin receptor CD71. In contrast, CD71 positive reticulocytes rarely contained Babesia nuclei and failed to express Babesia antigens. Accordingly, the frequency of YOYO-1 positive, CD71 negative cells strongly correlated with parasitemia defined as the frequency of infected red blood cells assessed on Giemsa stained blood smears. The absolute numbers generated by the two techniques were similar. Parasitemia was modest and transient in DBA/2 mice, but intense and sustained in C.B-17.scid mice. In both strains, parasitemia preceded reticulocytosis, but reticulocytes remained refractory to B. microti. In immunocompetent C.B-17 mice, reticulocytosis developed early, despite a marginal and short-lived parasitemia. Likewise, an early reticulocytosis developed in resistant BALB/cBy and B10.D2 mice.

Babesia microti Preferentially Reside in Mature Erythrocytes

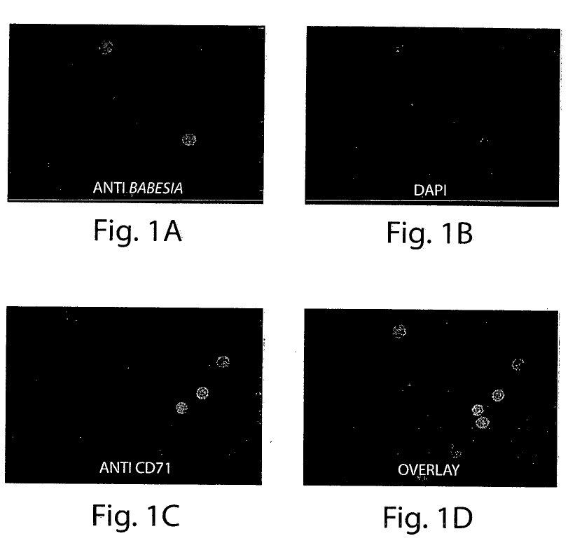

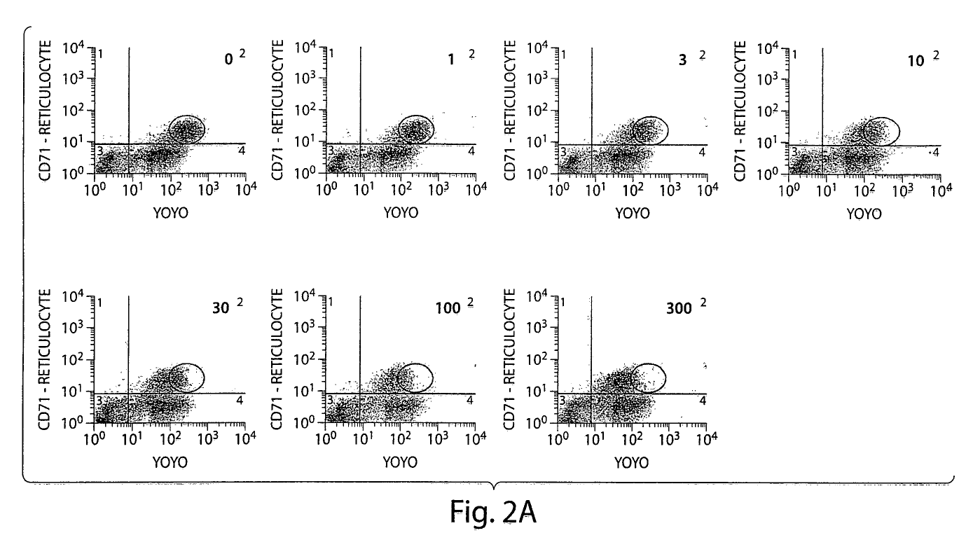

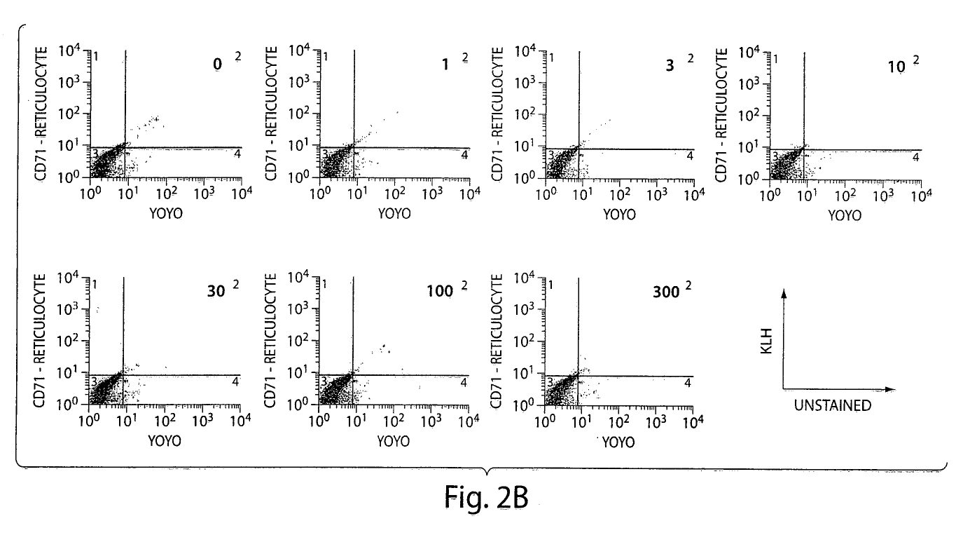

B. microti invasion of mature erythrocytes and/or immature reticulocytes was determined by obtaining blood cells from a C.B-17.scid mouse three months after the mouse was infected with 10⁵ pRBCs. As this strain lacks T and B cells, an intense parasitemia (circa 40% of infected red blood cells) persists during the second and third months post-infection. Blood cells were stained for CD71, the transferrin receptor found on most reticulocytes but not on mature erythrocytes. Parasite-derived nuclei were stained with DAPI, a DNA specific stain. Babesia antigens were revealed by a polyclonal antibody obtained from a Babesia-infected mouse. As shown in FIG. 1, DNA (blue) and Babesia antigens (red) were detected in CD71 negative but not in CD71 positive cells (green). Babesia antigens were detected at the membrane of infected erythrocytes and never co-localized in the cell with parasite-derived DNA. Thus, B. microti DNA and antigens are detected in mature erythrocytes. A flow cytometric assay was used for quantitative assessment of Babesia infected red blood cells. Blood cells were obtained from an infected C.B-17.scid mouse. Following fixation and permeabilization, cells were stained with YOYO-1, a cyanine dimer that binds both DNA and RNA. Whereas DAPI stained infected CD71 negative cells only (FIG. 1), YOYO-1 stained both CD71 negative and CD71 positive cells (FIG. 2). Since reticulocytes are rich in RNA, cells were treated with DNAse-free RNAse A. As the concentration of RNAse A was increased from 1 to 300 μg/ml, the intensity of YOYO-1 staining decreased in CD71 positive cells (FIG. 2). In contrast, YOYO-1 staining remained unchanged in CD71 negative cells. Thus, the flow cytometry assay corroborated the observations made by fluorescence microscopy, i.e., parasite-derived DNA accounts for the nucleic acid staining in erythrocytes, but not in reticulocytes.

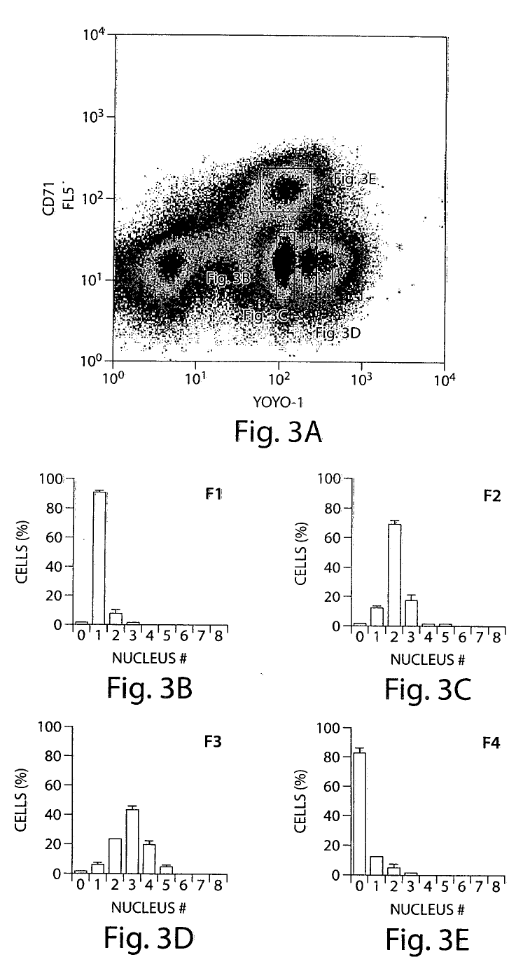

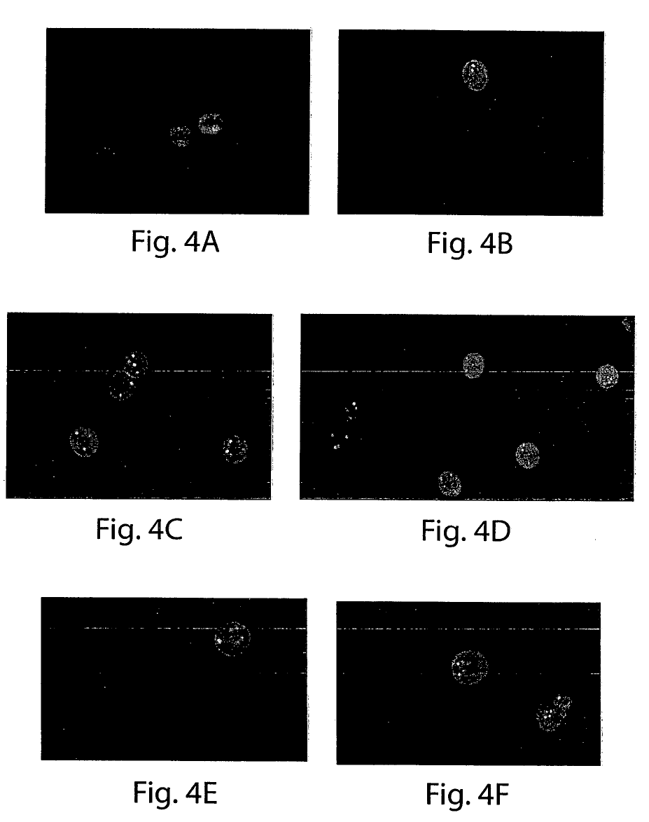

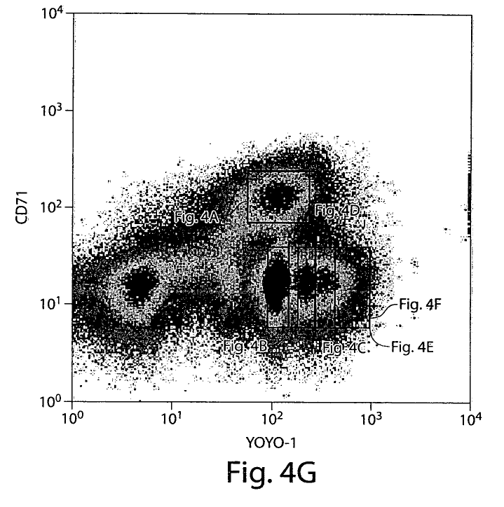

Red blood cells were sorted according to nucleic acid and CD71 staining (FIG. 3, central panel). Sorted cells were stained for the pan-erythroid surface marker TER119. When examined under fluorescent light (FIG. 4), the parasite nuclei appeared as green dots while the red blood cell membrane appeared red. Most CD71 negative cells with low YOYO-1 staining contained one nucleus (FIG. 3, fraction F1). As the intensity of YOYO-1 staining increased, the number of nuclei per cell increased (FIG. 3, fractions F2+F3). A greater total amount of nuclear material allowed a wider distribution of nucleus number per cell because nucleus size varied (FIG. 4, panels C-E). In some cells, the close proximity of nuclei revealed two daughter cells generated by binary fission (FIG. 4, panel B). In others, nuclei were arranged in a tetrad or Maltese cross (FIG. 4, panel F), a morphology pathognomonic of babesiosis (23). Scattered nuclei were frequent in CD71 negative cells with high YOYO-1 staining, suggesting multiple infections per cell. CD71 positive cells were sorted as a single fraction (F4) as they were stained homogenously by YOYO-1. Most CD71 positive cells (82%) contained no parasite nucleus whereas most of the remaining cells contained one nucleus only (FIG. 3, fraction F4). In cells devoid of a parasite nucleus, the YOYO-1 stain appeared as dim tiny green dots scattered uniformly throughout the cell (FIG. 4, panel A), confirming that YOYO-1 stains residual RNA left undigested by RNAse A. These observations indicate that i) B. microti has a predilection for mature erythrocytes and ii) nucleic acid staining in erythrocytes is a function of the number and size of parasite nuclei.

Reticulocytes Remain Refractory to B. microti During the Course of Infection in the Susceptible DBA/2 and C.B-17.scid Strains.

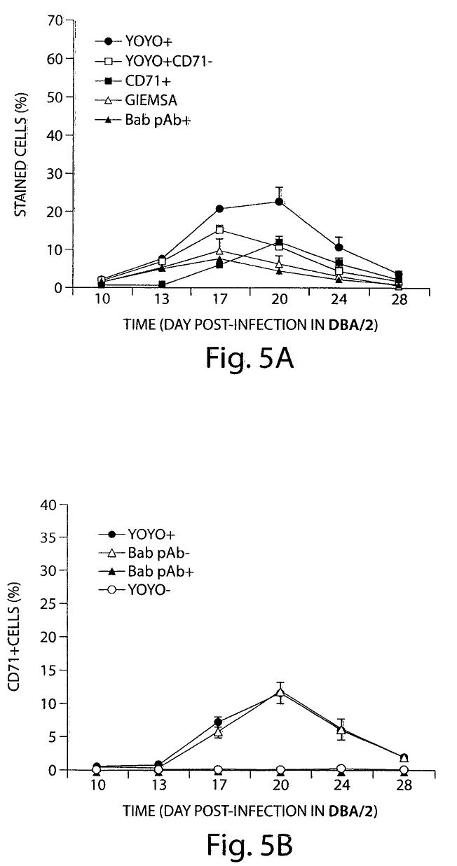

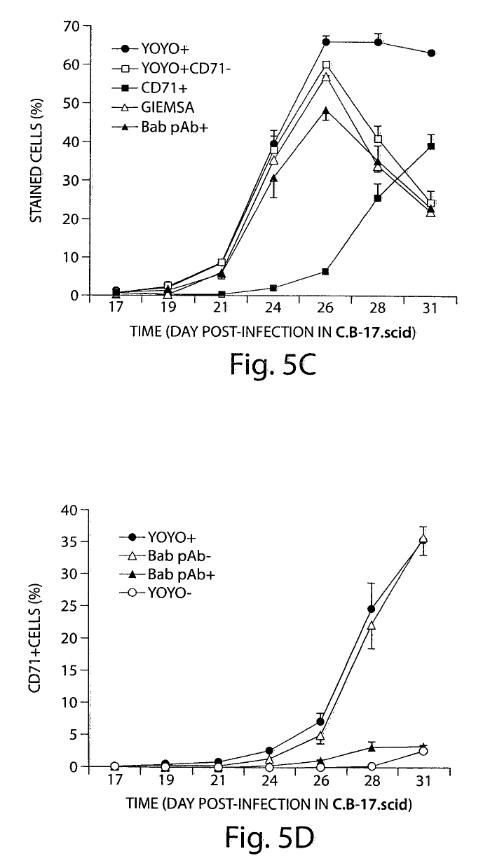

The host cell of choice changes during the course of B. microti infection. DBA/2 mice were inoculated with 10⁵ pRBCs and blood obtained at two-to-four day intervals (FIG. 5). A first drop of blood was dedicated to Giemsa staining on thin blood smears. A second drop was used for flow cytometric analysis of blood cells stained for nucleic acids, CD71 and Babesia antigens. Parasitemia (defined as the percent of infected red blood cells counted on Giemsa stained blood smears) started to rise on day 10, peaked on day 17 (10%), gradually decreased thereafter to become undetectable on day 28 (FIG. 5A). The frequency of red blood cells expressing Babesia antigens demonstrated similar kinetics. The rise in parasitemia was followed by a reticulocytosis that was detected on day 17, peaked on day 20, and gradually returned to basal levels thereafter. At each time point, nearly all CD71 positive cells were stained by YOYO-1, but were negative for Babesia antigens (FIG. 5B). There were rare CD71 positive cells that expressed Babesia antigens or did not stain with YOYO-1. Accordingly, the frequency of CD71 negative cells stained by YOYO-1 followed kinetics similar to those of cells expressing Babesia antigens and to that of parasitemia determined on Giemsa-stained smears.

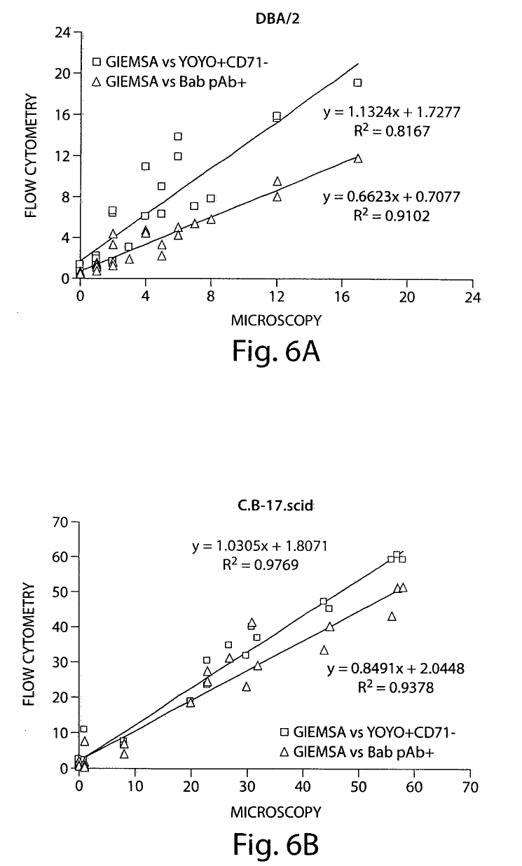

Parasitemia and reticulocytosis are moderate and reversible in DBA/2 mice. Reticulocyte infection with B. microti in hosts that develop a severe and sustained infection was determined. C.B-17.scid mice were infected with 10⁵ pRBCs. Parasitemia determined on Giemsa-stained blood smears rose on day 19 to reach a peak (57%) on day 26, and declined to moderate levels (22%) on day 31 (FIG. 5C). A similar time-course was observed for Babesia antigen-positive cells. In contrast, reticulocytosis trailed parasitemia by five days (FIG. 5C). Although CD71 positive cells represented some 40% of the blood cells on day 31, only 2% expressed Babesia antigens (FIG. 5D). As seen in DBA/2 mice, nearly all CD71 positive cells were stained by YOYO-1. Thus, even under conditions of sustained infection with high levels of parasitized red blood cells, reticulocytes are rarely infected. Correlation of the parasitemia detected on Giemsa-stained blood smears with the frequency of mature erythrocytes containing nucleic acids (YOYO+CD71− cells) as detected by flow cytometry over the course of infection was also determined. Microscopically determined parasitemia strongly correlated with the frequency of YOYO+CD71− cells in DBA/2 (r=0.90, P<0.001) and C.B-17.scid mice (r=0.99, P<0.001) (FIG. 6). These linear relationships had a slope of 1.13 and 1.03 in DBA/2 and C.B-17.scid mice, respectively, indicating that an increase in parasitemia translates into an increased frequency of YOYO+CD71− cells. Since the origins were 1.73 and 1.81 in DBA/2 and C.B-17.scid mice, respectively, the flow cytometric assay displayed a slight gain in sensitivity. The association between parasitemia and the frequency of cells expressing Babesia antigens was also examined. While correlations were strong in both DBA/2 (r=0.95, P<0.001) and C.B-17.scid mice (r=0.97, P<0.001), slopes were below 1. Likewise, the frequency of YOYO+CD71− cells strongly correlated with the frequency of Babesia antigen positive cells in DBA/2 (r=0.92, P<0.001) and C.B-17.scid mice (r=0.98, P<0.001), but slopes remained below 1. These results indicate that the polyclonal antibody directed against Babesia antigens detected fewer infected cells than the microscopic analysis of Giemsa-stained blood smears or the flow cytometry assay based on CD71 and nucleic acid staining.

Reticulocytosis is Induced in Mouse Strains Resistant to B. microti.

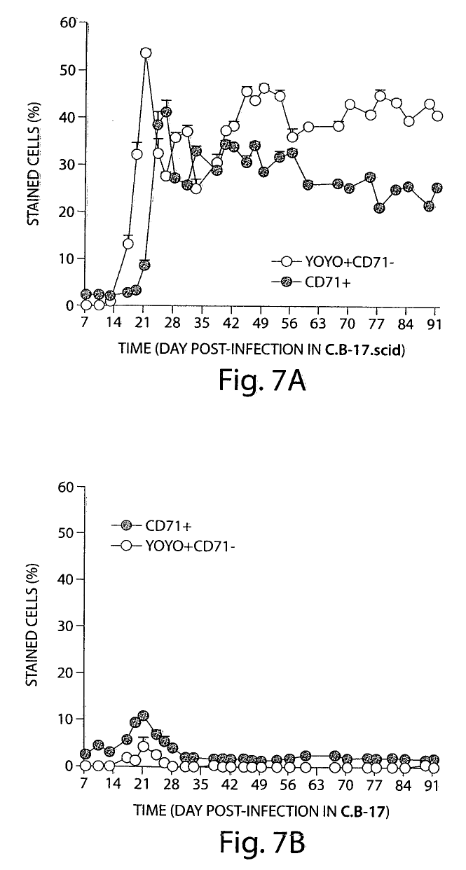

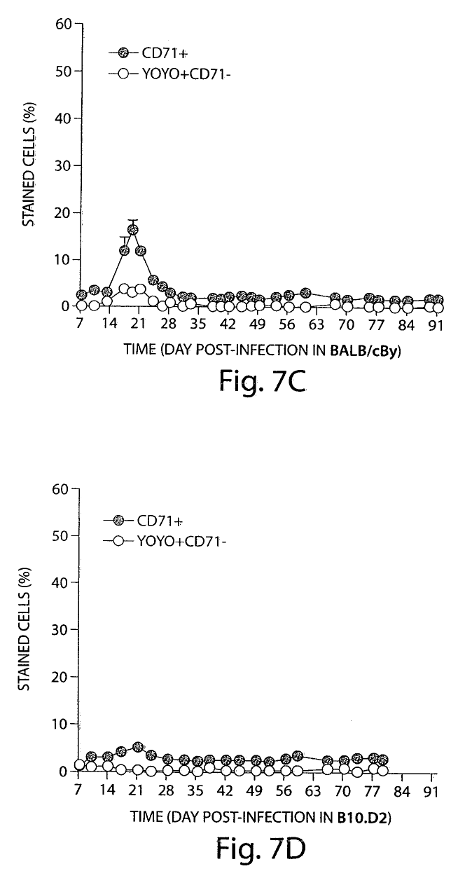

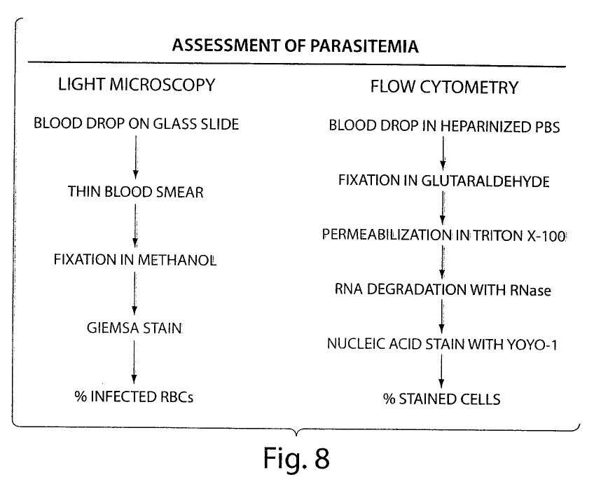

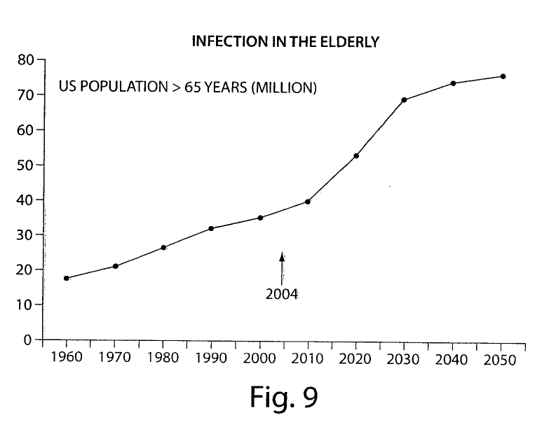

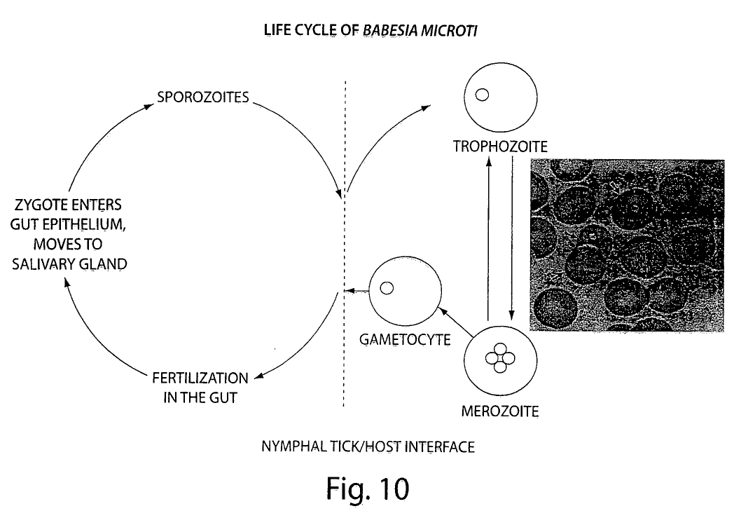

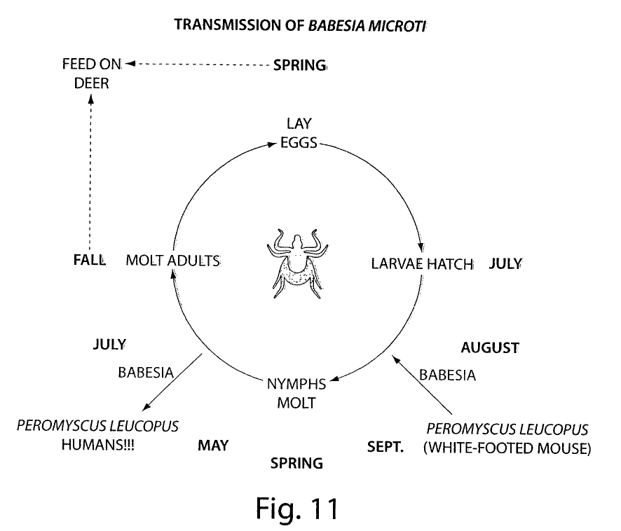

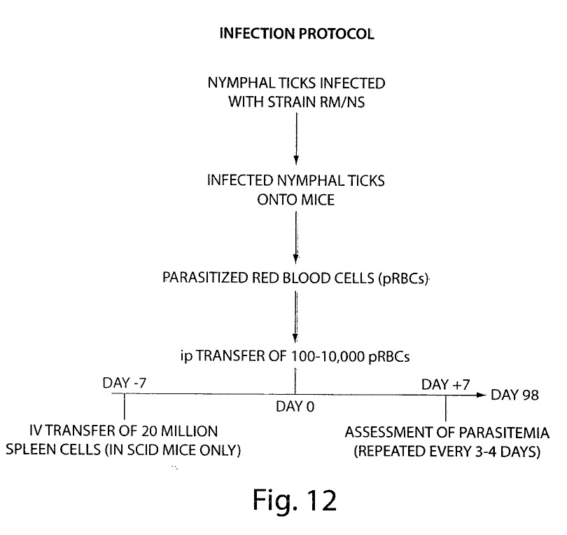

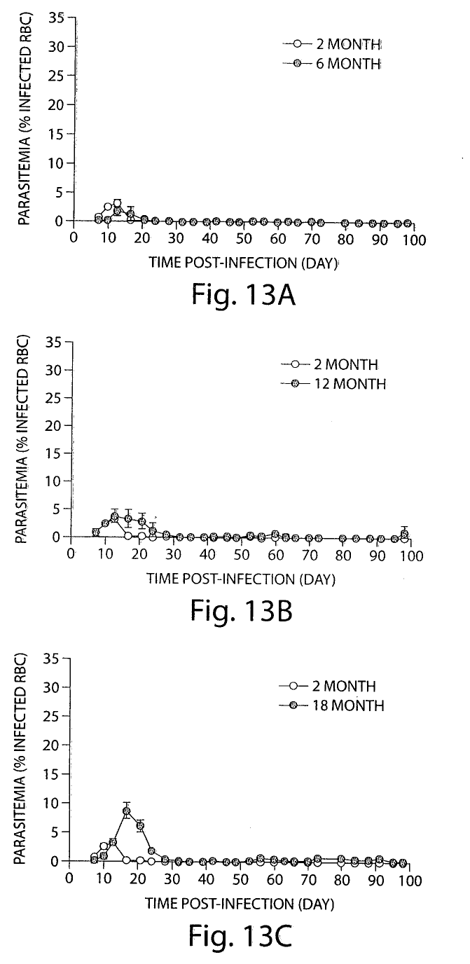

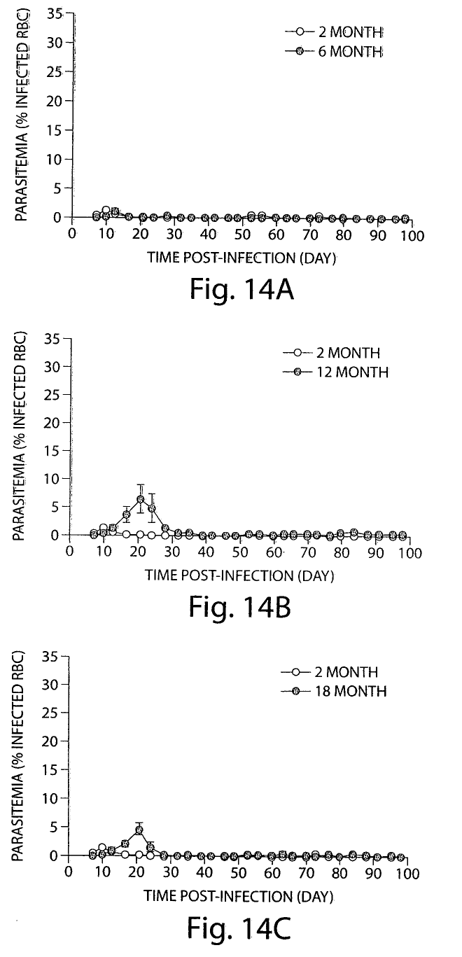

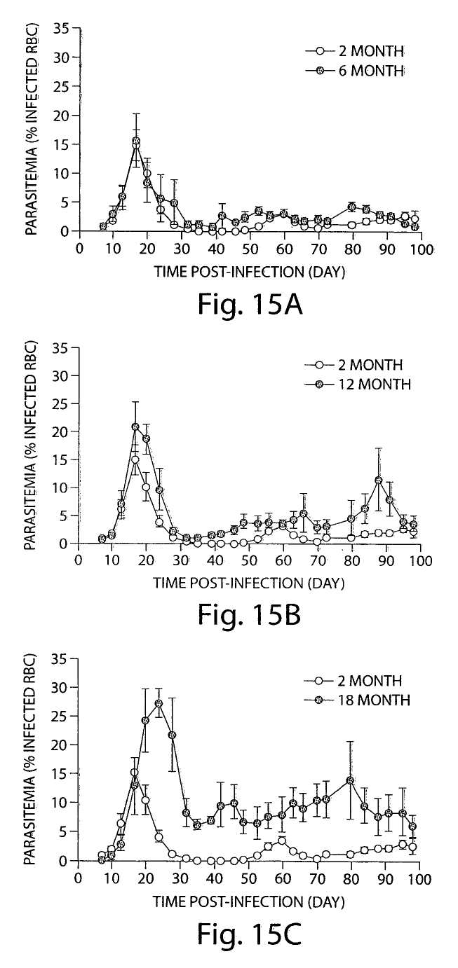

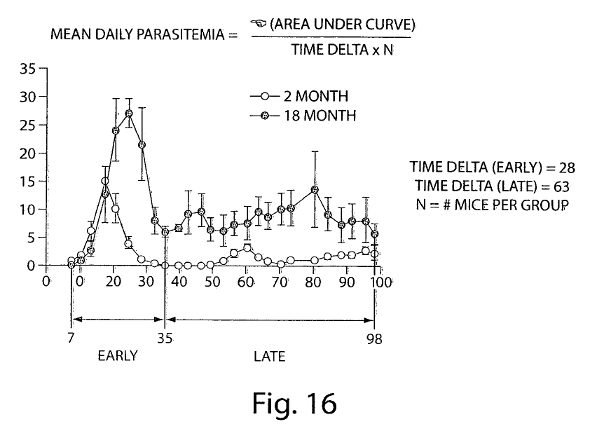

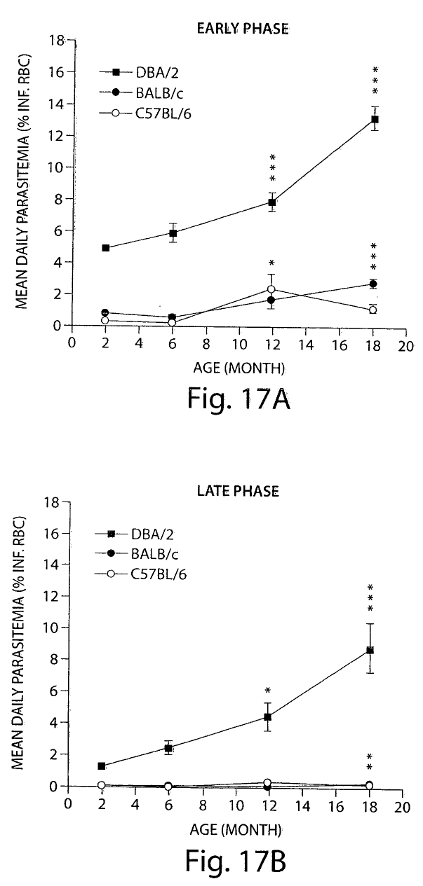

Infection of immunocompetent BALB/c mice with B. microti is followed by a low and short-lived parasitemia. In contrast, an intense and sustained parasitemia develops in infected C.B-17.scid mice. In addition to the spontaneous scid mutation, C.B-17.scid mice carry the Igh^{b} allele from the C57BL/Ka strain on an otherwise BALB/c background. The delayed reticulocytosis in C.B- 17.scid mice is a function of the genetic background as demonstrated by analysis of reticulocytosis and parasitemia in infected C.B-17 and C.B-17.scid mice (FIG. 7). Here, parasitemia was defined as the frequency of YOYO+CD71− cells. In C.B-17.scid mice (FIG. 7A), parasitemia rose on day 17, peaked for a first time on day 21, oscillated until day 45 to stabilize thereafter. Reticulocytosis was delayed as it rose on day 21 to stabilize on day 33. In C.B-17 mice (FIG. 7B), parasitemia was highest (4%) on day 21, but receded within five days. In these mice, reticulocytosis was early and transient (11% at peak). A similar pattern was seen in the BALB/cBy mice (FIG. 7C). Parasitemia reached a modest plateau (3-4%) between days 17 and 21, whereas reticulocytosis peaked at 16% on day 19. Parasitemia was monitored in C57BL/10 mice congenic for the MHC haplotype H^{2d} of the DBA/2 strain (FIG. 7D). B10.D2 mice were highly resistant to infection, but developed a modest and short-lived reticulocytosis that also peaked (5%) on day 21. Therefore, while the intensity of reticulocytosis varies with the degree of parasite burden, the timing of reticulocytosis appears to differ between strains, i.e., parasitemia precedes reticulocytosis in the susceptible DBA/2 and C.B-17.scid strains whereas parasitemia and reticulocytosis develop simultaneously in resistant strains such as C.B-17, BALB/cBy and B10.D2.

Diagnosis of Red Blood Cell Protozoal Parasites

Babesiosis has been routinely diagnosed by microscopic analysis of Giemsa-stained thin blood smears. Additional tests include the indirect fluorescent antibody test (IFAT), ELISA and PCR. Because B. divergens infections are already fulminant when antibody titers rise, IFAT and ELISA are of limited diagnostic value. In B. microti infections, serology is positive prior to the onset of parasitemia. However, since antibodies persist even after parasites are cleared, IFAT and ELISA fail to distinguish an active from a recent infection. PCR detects the overall presence of babesial DNA in a blood sample, but provides no information on the number of infected red blood cells or on the number of parasites per cell. Flow cytometric assays avoid these limitations. Hydroethidine has been used in studies of B. bovis and B. canis infected red blood cells. The assay relies on the uptake and metabolic conversion of hydroethidine into ethidium by live parasites. Because conversion does not occur in reticulocytes, this assay excludes from detection those Babesia species that have a predilection for reticulocytes, such as B. gibsoni.

The methods of the invention provide solutions to many of the drawbacks of earlier methods. The methods detect parasite DNA in red blood cells such as mature erythrocytes and reticulocytes. In testing reticulocytes, reticulocyte RNA is first digested using a RNA-degrading composition such as an RNase enzyme. The diagnostic assay utilizes a strong fluorescence signal generated by a sensitive nucleic acid dye such as YOYO-1. In diagnosing infection with B. microti, the assay distinguishes reticulocytes from erythrocytes on the basis of transferrin receptor surface expression. Compared to the traditional microscopy analysis, the assay does not rely on the microscopist eye to make a decision, allows the detection of red blood cells at different stages based on surface marker, and is amenable to high-throughput.

Using well-recognized mouse models of babesiosis, the studies indicated that reticulocytes are rarely infected with the human pathogen B. microti. The frequency of YOYO-1 positive erythrocytes is a surrogate measure of parasitemia in infections with Babesia species that do not invade reticulocytes. The assay is modified by the addition of an RNA-digestion step to diagnose infection with other Babesia species or other protozoal parasites that do infect (or reside in) reticulocytes or parasites that infect (or reside in) both reticulocytes and mature erythrocytes.

Prior to the invention, information regarding the tropism of Babesia species for erythrocytes or reticulocytes was scarce. Whether B. microti has a differential tropism was unknown. Data indicated that BALB/c.scid mice showed exquisite susceptibility to B. microti, and in this model of severe chronic infection, parasitemia sharply increased to reach high and sustained levels within a month. Despite an intense parasite burden and a sustained reticulocytosis, reticulocytes have now been shown to be rarely infected. Reticulocytes were sorted on the basis of high nucleic acid content and high CD71 surface expression. When red blood cells were collected in the second month of sustained and persistent parasitemia, only 12% of CD71 positive cells contained one parasite nucleus while less than 5% contained two nuclei. CD71 positive cells that contained three nuclei or more were very rare. However, even in the absence of parasite nuclei, CD71 positive cells had an intense staining of nucleic acids by YOYO-1. This staining appeared in the form of tiny dim dots scattered uniformly throughout the CD71 positive cell, indicating that YOYO-1 is powerful enough to detect residual RNA left undigested by treatment with DNAse-free RNAse A. As these dots are numerous, the fluorescence emitted on a cell basis is equivalent to that emitted by one or two parasite nuclei typically found in mature erythrocytes (CD71 negative cells). In these cells, parasite nuclei were stained by YOYO-1 (or DAPI) as large dots. The low frequency of parasite-derived nuclei in CD71 positive cells in a model of severe chronic infection indicates that reticulocytes are not the host cell of choice for invasion by, and budding of B. microti.

As YOYO-1 stains nucleic acids with no sequence specificity, the design of the flow cytometry assay for B. microti is made possible by the absence of mammalian nuclei in reticulocytes and erythrocytes. To ascertain that YOYO-1 stained erythrocytes are infected with B. microti, we assessed the frequencies of Babesia antigen expressing cells in C.B-17.scid and DBA/2 mice during the first month post-infection. For both strains, the frequencies of Babesia antigen expressing cells strongly correlated with parasitemia values determined on Giemsa stained blood smears.

Babesia antigens were detected at the surface of infected erythrocytes, but not at the parasite itself. Flow cytometric analysis of infected red blood cells indicated that Babesia antigens are localized to the inner leaflet of the red blood cell membrane since they are not detected in unfixed (and non-permeabilized) cells. The results described above confirm that YOYO-1 stained erythrocytes are infected with B. microti, and indicate that parasitized erythrocytes, in their majority, present Babesia antigens at their cytoplasmic membrane.

Defining the host cells in babesiosis is critical to the understanding of resistance and/or susceptibility. In a mouse model of malaria infection using P. yoelii which preferentially infects reticulocytes, IFN-γ confers protection by suppressing erythropoiesis, i.e., by decreasing the numbers of circulating reticulocytes. Conversely, the failure of IFN-γ to prevent or reduce infection of mice with P. vinckei petteri has been attributed to the fact that this parasite invades solely mature red blood cells. Mature erythrocytes have now been identified as the main host cell of B. microti in two susceptible mouse strains. Because reticulocytes remain rarely infected throughout the course of severe infection, an early erythropoiesis may contribute to resistance by increasing the frequency of non-host reticulocytes while decreasing the frequency of erythrocytes, the host cell. A significant and short-lived reticulocytosis was concomitant to a modest, if not marginal parasitemia in mice of two resistant strains, namely BALB/cBy and B10.D2. Likewise, the kinetics of reticulocytosis and parasitemia overlapped in the resistant C.B-17 mice (on a BALB/c background). In striking contrast, reticulocytosis was delayed in C.B-17.scid mice which lack peripheral T and B cells, and displayed an exquisite susceptibility to infection with B. microti. Likewise, the susceptible DBA/2 strain developed a delayed reticulocytosis. Thus, the delayed reticulocytosis in susceptible strains appears to result from an inefficient or deficient immune response, rather than from allelic variations that would directly affect the generation of reticulocytes.

B. gibsoni is a species that preferentially infects reticulocytes. Species that infect bovine animals (e.g., B. bigemina, B. bovis, B. divergens) and species that infect canine animals (e.g., B. canis) also have a preference for infecting immature erythrocytes or reticulocytes. Babesia is endemic in certain regions of the world such as the US, Brazil, Argentina, Mexico and central Europe. The assays described herein are used to screen for and identify infected animals for veterinary use and in livestock animals, particularly in cattle in major beef producing markets such as Brazil, Argentina, Uruguay, and the United States.

Studies of B. gibsoni infection have shed some light on how an intraerythrocytic pathogen can hijack erythropoiesis. B. gibsoni inhibits the activity of 5′-nucleotidase, an enzyme that degrades ribosomal RNA in reticulocytes. By doing so, B. gibsoni prevents the maturation of reticulocytes. The reduced 5′-nucleotidase activity leads to an accumulation of pyrimidine and purine nucleotides, such as cytidine 5′-monophosphate and inosine 5′-monophosphate. The former inhibits parasite replication and retards reticulocyte maturation whereas the latter inhibits parasite replication. As reticulocyte lifespan expands and parasite growth is curtailed, this scenario favors both parasite and host survival. Because B. microti preferentially resides in mature erythrocytes, the regulation of erythropoiesis, likely differ. By delaying the generation of reticulocytes, B. microti may protect the mammalian host from an overwhelming parasitemia that would lead to massive hemolysis, and ultimately compromise the survival of the host and the parasite itself.

The assays described herein are useful in human and veterinary medicine to diagnose infection with Babesia microti primarily infects mature erythrocytes using a flow cytometric assay that relies on the detection of nucleic acids by the sensitive dye YOYO-1, and on the identification of reticulocytes as CD71 positive cells. With the addition of an RNase degradation step, the flow cytometry based assays are also useful to diagnose infection of CD71 positive reticulocytes with parasites, such as malaria-causing protozoan pathogens (for example, Plasmodium sp.), based on the presence of DNA in those cells. The flow cytometric assays are also useful to monitor efficacy of therapy by detecting a reduction in parasite DNA over time over the course of therapeutic intervention.

### EQUIVALENTS

Although particular embodiments have been disclosed herein in detail, this has been done by way of example for purposes of illustration only, and is not intended to be limiting with respect to the precise form of the disclosed invention or to the scope of the appended claims that follow. In particular, it is contemplated by the inventors that various substitutions, alterations, and modifications may be made to the invention without departing from the spirit and scope of the invention as defined by the claims. Various alterations and modifications of the invention are believed to be a matter of routine for a person of ordinary skill in the art with knowledge of the embodiments described herein. Other aspects, advantages, and modifications considered to be within the scope of the following claims.

1. A method for diagnosing the presence of a parasitic microorganism, comprising determining a fraction of DNA-containing, transferrin receptor-negative cells in a sample of mammalian red blood cells in fluid phase, wherein an increase in said fraction compared to a normal fraction indicates that a mammal is infected with said parasitic microorganism.

2. The method of claim 1, wherein said parasitic organism is a Babesia sp.

3. The method of claim 1, wherein said parasitic organism is Babesia microti.

4. The method of claim 1, wherein said parasitic organism is Plasmodium sp.

5. The method of claim 1, wherein said parasitic organism is Bartonella sp.

6. The method of claim 1, wherein said sample comprises erythrocytes.

7. The method of claim 1, wherein said sample contains a population of reticulocytes

8. The method of claim 7, wherein said method comprises identifying said population by contacting said population with a transferrin receptor ligand labeled with a detectable marker.

9. The method of claim 1, wherein said method comprises contacting said mammalian blood cells with an RNA-degrading composition.

10. The method of claim 1, wherein said method comprises detecting expression of a CD71 antigen on the surface of said red blood cell.

11. The method of claim 1, further comprising contacting said mammalian red blood cells with a CD71-specific antibody.

12. The method of claim 1, wherein said method further comprises fixing said sample of mammalian red blood cells.

13. The method of claim 8, wherein said fixing step comprises contacting said sample with glutaraldehyde.

14. The method of claim 1, wherein said method further comprises permeabilizing said sample of mammalian red blood cells.

15. The method of claim 14, wherein said permeabilizing step comprises contacting said sample with a detergent.

16. The method of claim 1, wherein said method comprises determining said fraction of DNA-containing mammalian red blood cells by contacting said sample of red blood cells with a fluorescent nucleic acid stain.

17. The method of claim 16, wherein said nucleic acid stain is a dimeric cyanine nucleic acid stain.

18. The method of claim 16, wherein said nucleic acid stain is YOYO-1.

19. The method of claim 16, wherein said nucleic acid stain is selected from the group consisting of propidium iodide, thiazole orange, SYBR Green I, SYTOX Green, POPO-1, BOBO-1, YOYO-1, POPO-3, LOLO-1, BOBO-3, YOYO-3, TOTO-3, TOPRO3, and PicoGreen.

20. The method of claim 1, wherein said detecting step is carried out by flow cytometry.

21. A method of diagnosing the presence of a parasitic organism, comprising contacting a sample of red blood cells with an RNA-degrading composition and determining a fraction of DNA-containing red blood cells in fluid phase, wherein an increase in said fraction compared to a normal fraction indicates that a mammal is infected with said parasitic microorganism.

22. A kit for diagnosis of a parasitic microorganism in a mammalian red blood cell, comprising a ligand that binds to an epitope of the transferrin receptor and a composition that binds to nucleic acids.

23. The kit of claim 22, wherein said transferrin receptor is CD71.

24. The kit of claim 22, wherein said ligand is an antibody.

25. The kit of claim 22, wherein said ligand and said composition comprise different fluorescent probes.

26. The kit of claim 22, wherein said composition is YOYO-1.
